(19) United States
(12) Patent Application Publication
(10) Pub. No.: US 2021/0147990 A1
(43) Pub. Date: May 20, 2021

(54) IN VITRO PRODUCTION OF HYDROGEN UTILIZING HYDROGENASE

(71) Applicant: Alliance for Sustainable Energy, LLC, Golden, CO (US)

(72) Inventors: Maria Lucia GHIRARDI, Lakewood, CO (US); Drazenka SVEDRUZIC, Golden, CO (US); Judith Cecilia VIDAL, Littleton, CO (US)

(21) Appl. No.: 17/046,827

(22) PCT Filed: Apr. 8, 2019
(86) PCT No.: PCT/US19/26287

§ 371 Date: Oct. 12, 2020

Related U.S. Application Data
(60) Provisional application No. 62/656,689, filed on Apr. 12, 2018.

(51) Int. Cl. C25B 11/075; C25B 1/04; C12N 9/04

(52) U.S. Cl. CPC C25B 11/075; C12N 9/0006; C25B 1/04

(57) ABSTRACT

The present application overcomes the issue of the low yields of biological hydrogen production by cells by utilizing purified hydrogenase enzymes in vitro. Two methods are considered. The major advantage of these approaches is that they by-pass the mechanisms that limit the rates of intact organisms, thus achieving rates much closer to the actual capacity of the enzyme.

### CROSS-REFERENCE TO RELATED APPLICATION

This application claims the benefit of U.S. Provisional Application 62/656,689 filed Apr. 12, 2018, the contents of which are incorporated herein by reference in their entirety.

The United States Government has rights in this invention under Contract No. DE-AC36-08GO28308 between the United States Department of Energy and the Alliance for Sustainable Energy, LLC, the Manager and Operator of the National Renewable Energy Laboratory.

### BACKGROUND

The production of renewable hydrogen to support a fuel-cell-based economy is an area of increasing interest for many researchers. Many existing technologies are either based on expensive and/or high energy requiring processes or on industrial fossil fuels (e.g., methane reformation). Biological hydrogen production systems also exist that utilize microorganisms that express hydrogenase enzymes capable of hydrogen generation from organic wastes. Such systems may involve the direct in vivo production of hydrogen by microorganisms exposed to waste streams.

The foregoing examples of the related are and limitations therewith are intended to be illustrative and not exclusive. Other limitations of the related art will become apparent to those of skill in the art upon a reading of the specification and a study of the drawings.

### SUMMARY

An aspect of the present disclosure is a method comprising depositing a hydrogenase on an electrode, immobilizing the hydrogenase on the electrode using a matrix, supplying an electric current through the electrode to the hydrogenase, adding acid to the hydrogenase, and supplying an inert gas to the hydrogenase. In some embodiments, the electrode may be a carbon electrode. In some embodiments, the carbon electrode may consist substantially of glassy carbon, pyrolytic graphite, or carbon felt. In some embodiments, the carbon electrode may be radio translucent.

In some embodiments, the hydrogenase may be a purified algal hydrogenase. In some embodiments, the purified algal hydrogenase may be from Chlamydomonas reinhardtii. In some embodiments, the matrix may be electroconductive. In some embodiments, the acid may be sulfuric acid. In some embodiments, the inert gas may be argon. In some embodiments, a biofilm may also be used to immobilize the hydrogenase.

An aspect of the present disclosure is a device comprising an electrode, a hydrogenase immobilized directly on the electrode, a matrix encapsulating the hydrogenase, and a chamber, wherein the hydrogenase, the matrix, and the electrode are positioned within the chamber, the electrode is attached to a power source, and the chamber has at least one vent. In some embodiments, the electrode may be a carbon electrode. In some embodiments, the carbon electrode may consist substantially of glassy carbon, pyrolytic graphite, or carbon felt. In some embodiments, the carbon electrode may be radio translucent. In some embodiments, the hydrogenase is purified algal hydrogenase. In some embodiments, the purified algal hydrogenase is from Chlamydomonas reinhardtii. In some embodiments, the matrix is electroconductive. In some embodiments, the acid is sulfuric acid. In some embodiments, the inert gas is argon. In some embodiments, the matrix is a biofilm.

The hydrogenase enzyme may be anaerobically stabilized on the electrode surface for a period of time prior to the current being supplied to the carbon electrode. This period may be less than one hour.

In some embodiments of the present invention, the initial pH of the electrode solution may be approximately 6.5 to 7.5. The pH of the system may be maintained using an acidic solution. The electric current supplied to the hydrogenase enzyme through the carbon electrode may have a voltage of approximately 0.65-7.5 V.

A further aspect of the present invention is a method, where the method includes immobilizing a hydrogenase enzyme in an electroconductive matrix prior to depositing it on a carbon electrode. The method includes using a matrix to maintain the hydrogenase enzyme in close association with the carbon electrode. The hydrogenase enzyme is maintained at a consistent pH level by adding an acidic solution to the electrode solution. The acidic solution may be a strong acid and/or a mineral acid. In some embodiments, the acidic solution may be comprising of hydrochloric acid.

The matrix may be alginate or other conductive polymers. The hydrogenase enzyme may not be in direct contact with the carbon electrode. The matrix may be in direct contact with the carbon electrode. The matrix may render the hydrogenase enzyme immobile. The hydrogenase enzyme may be anaerobically stabilized for a period of time prior to the current being supplied to the carbon electrode. The period of anaerobic incubation may vary.

In some embodiments of the present invention, the electric current supplied to the hydrogenase enzyme through the carbon electrode may have a voltage of approximately 6.5 to 7.5V. The voltage may be provided by renewable energy sources such as solar or wind power generation. The pH of the system may be maintained using an acidic solution.

### BRIEF DESCRIPTION OF THE DRAWINGS

Exemplary embodiments are illustrated in referenced figures of the drawings. It is intended that the embodiments and figures disclosed herein are considered to be illustrative rather than limiting.

FIG. 1 illustrates the hydrogenase enzyme and electrode interface according to some embodiments of the present invention.

FIG. 2 illustrates a matrix separating the hydrogenase enzyme and electrode, according to some embodiments of the present invention.

FIG. 3 illustrates a flowchart of a method as described by some embodiments of the present disclosure.

FIG. 4 illustrates chances in current density as a function of voltage for one testing of the present disclosure occurring on Mar. 2, 2017.

FIG. 5 illustrates current density as a function of voltage for one testing of the present disclosure occurring on Apr. 12 and 13, 2017.

FIG. 6 illustrates current density as a function of voltage for one testing of the present disclosure occurring on Mar. 22, 2017.

FIG. 7 illustrates current density as a function of time for one testing of the present disclosure occurring on Apr. 13, 2017.

FIG. 8 illustrates current density as a function of voltage for one testing of the present disclosure.

FIG. 9 illustrates current density as a function of time for one testing of the present disclosure occurring on Apr. 28, 2017.

FIG. 10 illustrates current density as a function of time for one testing of the present disclosure.

### REFERENCE NUMBERS

100 system
105 carbon electrode
110 purified algal hydrogenase enzyme
120 matrix
200 system
230 biofilm
300 method
301 step one
302 step two
303 step three
304 step four
305 step five

### DETAILED DESCRIPTION OF SOME EMBODIMENTS

The present disclosure may address one or more of the problems and deficiencies of the prior art discussed above. However, it is contemplated that some embodiments as disclosed herein may prove useful in addressing other problems and deficiencies in a number of technical areas. Therefore, the embodiments described herein should not necessarily be construed as limited to addressing any of the particular problems or deficiencies discussed herein. Embodiments discussed herein are directed to the production of hydrogen by purified algal hydrogenase enzymes used in vitro.

Disclosed herein are methods and systems for producing hydrogen gas utilizing the hydrogenase enzyme deposited on the surface of a carbon electrode. The methods and systems describe in vitro systems for producing hydrogen gas in a renewable manner. Some embodiments describe routing a current through the hydrogenase enzyme, while others describe embedding the hydrogenase in either a thin alginate film or alginate beads.

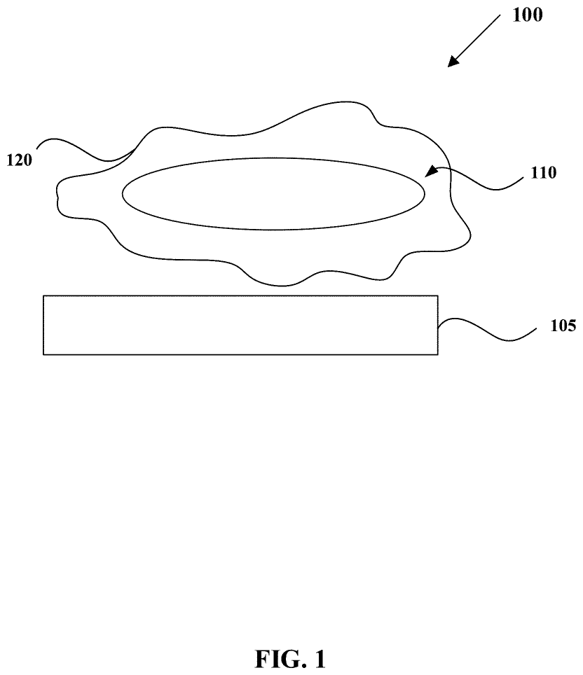

FIG. 1 illustrates the hydrogenase enzyme and electrode interface according to some embodiments of the present invention. As shown the system for producing hydrogen 10 includes purified algal hydrogenase 110 is deposited directly on a carbon electrode 105 according to exemplary embodiments of the present invention. The hydrogenase 110 may be suspended within a matrix 120. The matrix 120 may be electroconductive. The matrix 120 may be in direct contact with the carbon electrode 105. The hydrogenase enzyme may be a Chlamydomonas reinhardtii hydrogenase or related hydrogenase. The carbon electrode may be substantially composed of glassy carbon, pyrolytic graphite, or carbon felt or other polymers. The hydrogenase enzyme may be anaerobically incubated for a period of time prior to the current being supplied to the carbon electrode. The period of anaerobic incubation may be greater than one hour, one hour, or less than one hour.

To produce hydrogen, the hydrogenase 110 may receive current from the carbon electrode 105, which may be routed through the matrix 120. The current may be provided over the course of several minutes or several hours. The current may be provided at a steady pace or may be provided at varying levels while it is provided to the hydrogenase 110. In some embodiments an ammeter may be used to measure the current routed through the hydrogenase 110.

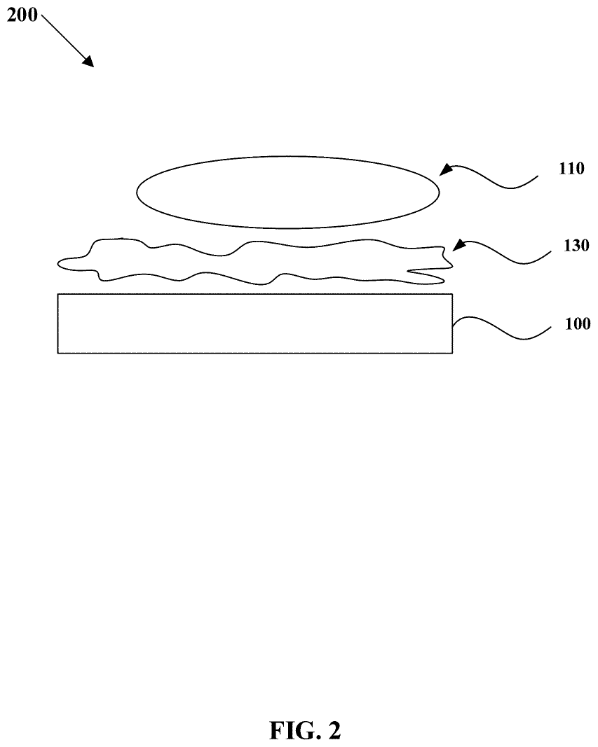

FIG. 2 illustrates a purified algal hydrogenase 110 suspended in a biofilm 230. The biofilm 230 may be in direct contact with the electrode 105. The biofilm 230 may prevent the hydrogenase 110 from coming in direct contact with the electrode 105. The system 200 may be maintained at a constant pH. A constant pH may be maintained by slowly adding an acidic solution.

In vitro hydrogen production has many advantages over the use of in vivo hydrogen production. These advantages include the absence of limitations placed by the microorganism's metabolism and the possibility of achieving high selectivity when presented with a single feedstock. In some embodiments, the present disclosure utilizes in vitro hydrogen production.

It has been shown that immobilization of microorganisms within biofilms results in decreased inactivation of their Hz-producing properties. Inactivation of purified hydrogenases can be prevented by immobilization directly onto electrode surfaces or within various polymer matrices.

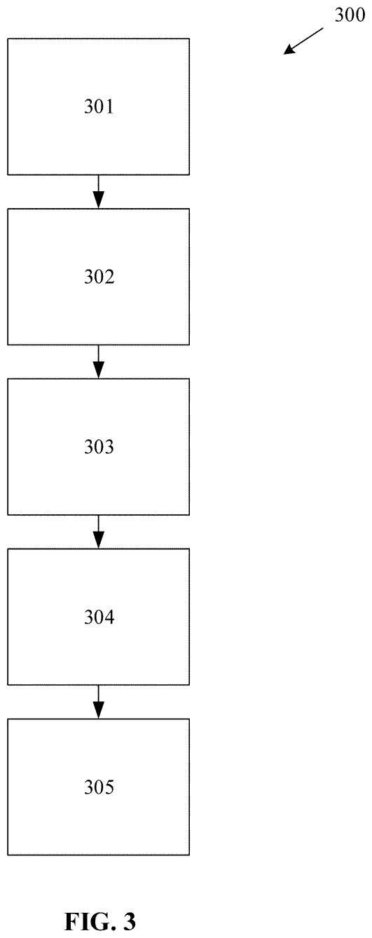

FIG. 3 illustrates a flowchart of a method as described by some embodiments of the present disclosure. The method of producing hydrogen 300 begins with a first step 301. The first step 301 includes depositing hydrogenase on an electrode. The hydrogenase may be algal in nature. The electrode may be made of glassy carbon, pyrolytic graphite, or carbon felt. The electrode may be radio translucent.

The second step 302 in the method of producing hydrogen 300 includes immobilizing the hydrogenase on the electrode using a matrix. In some embodiments the matrix may be electroconductive.

The third step 303 of the method of producing hydrogen 300 includes supplying an electric current to the system using the electrode. In some embodiments the current may be supplied at a steady rate. In other embodiments the current may be supplied at an increasing rate (i.e., the amount of current supplied increases as time goes on). In still other embodiments, a combination of steady application of current and changing levels of current may be used.

The fourth step 304 of the method of producing hydrogen 300 includes adding acid to the hydrogenase to maintain the pH of the system. In some embodiments, the amount of acid added may depend on the amount of current supplied to the hydrogenase.

The fifth step 305 of the method of producing hydrogen 300 includes purging the produced hydrogen gas with another gas. In some embodiments, the purging gas may be an inert gas. The inert gas may be argon, neon, helium, or another inert gas or combination thereof.

In some embodiments, step two 302 may occur before step one 301. That is, in some embodiments the hydrogenase may be immobilized using a matrix prior to being deposited on an electrode.

The matrix may encapsulate the hydrogenase and may act as a barrier between the hydrogenase and the electrode. The matrix may be in direct contact with the electrode and the hydrogenase, but the hydrogenase and the electrode may not be in direct contact with each other. The matrix may be made of alginate or other conductive fibers. In some embodiments the matrix may be a biofilm.

Three types of electrode materials were tested: glassy carbon, pyrolytic graphite, and carbon felt. Some carbon activation techniques were applied. Unless otherwise noted, experiments were done at pH 7.5. The voltage provided by the carbon electrode may be continuous.

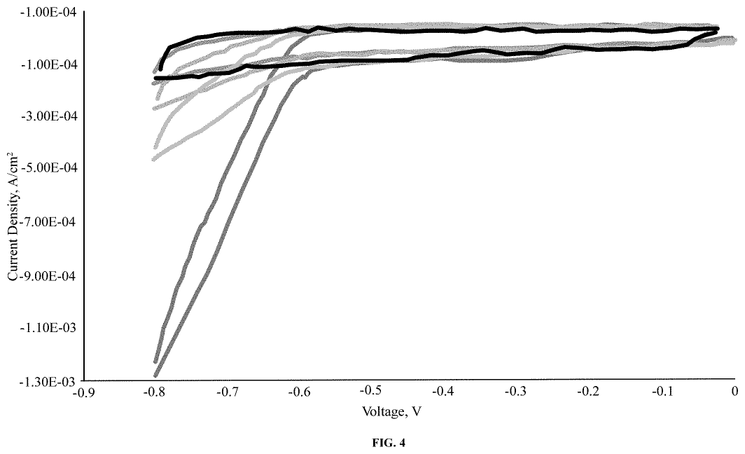

FIG. 4 illustrates chances in current density as a function of voltage for one testing of the present disclosure occurring on Mar. 2, 2017. In this test, a glassy carbon wet electrode (GCWE) was used. A hydrogenase was placed on the GCWE and a current was supplied to the electrode. A matrix was utilized to keep the hydrogenase in place on the GCWE while the current was supplied to the electrode. The current was supplied at a steady rate of −0.75V, however, other current levels could be used.

As shown in FIG. 4, using the glassy carbon electrode, the hydrogenase enzyme activity decreased over time under continuous exposure to −0.75V. Longer measurements, over the course of 20 hours, resulted in stable current densities below 0.07 μA/cm². FIG. 4 illustrates chances in current density as a function of voltage for one testing of the present disclosure. Changes in current density as a function of time, using hydrogenase enzyme immobilized on a glassy carbon electrode for 30 minutes, at which point measurements were taken. The initial pH was 7.5.

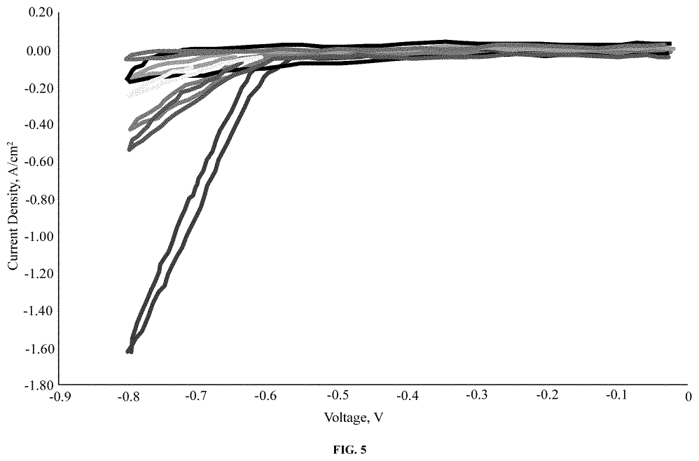

FIG. 5 illustrates current density as a function of voltage for one testing of the present disclosure occurring on Apr. 12 and 13, 2017. In this test, a glassy carbon (GC) electrode was used. Chrono-amperogram of hydrogenase enzyme immobilized onto a carbon felt electrode surface; the experiment started at pH 6.82 and acid was added at different points to determine the effect of a more acidic environment on current densities. The experiment lasted a total of 8 hours and ended with a pH value of 3.21. As shown in FIG. 5, high enzyme activity lasted for at least 4 hours before decreasing below 600 μA/cm² as the pH levels were decreased to 4.5.

In FIG. 5, an increase in pH from 6.82 to 6.86 during the initial 2 hours of the measurement reflects the production of hydrogen by the hydrogenase enzyme. Addition of acid at various points results in apparent increased deactivation rates (notice the slopes of the curves after each addition), suggesting increased hydrogenase enzyme sensitivity under lower pH.

The best results and indicates the maximum capability of the system to produce hydrogen gas during a period of 50 hours using a carbon felt electrode with an “exposed” electrode geometric surface of 1.53 cm².

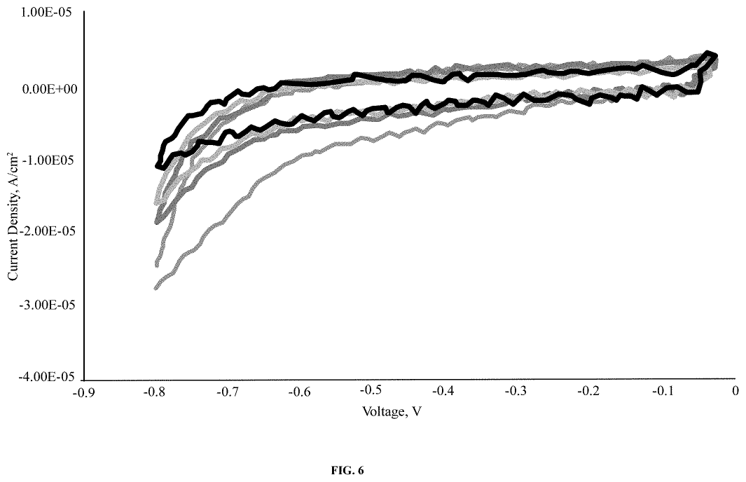

FIG. 6 illustrates current density as a function of voltage for one testing of the present disclosure occurring on Mar. 22, 2017. In this experiment, a pyrolytic graphite (PG) electrode was used. A hydrogenase was attached to the electrode using an electroconductive matrix.

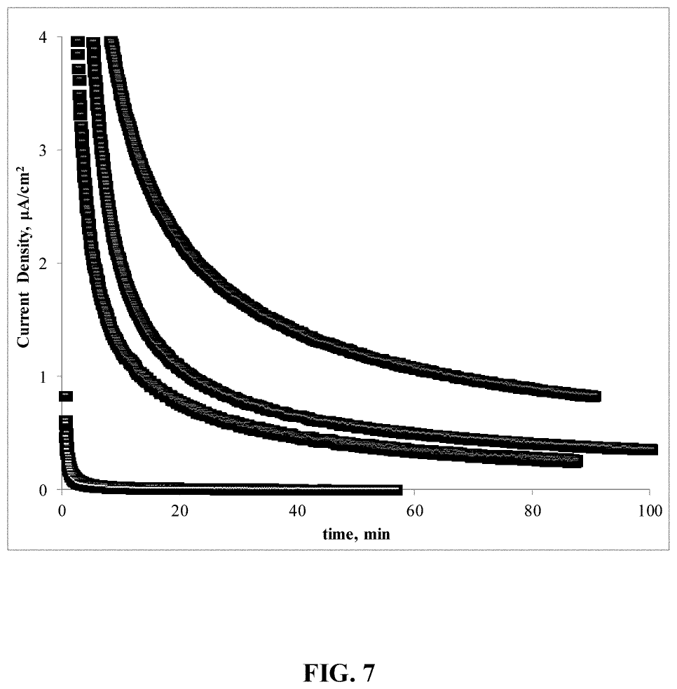

FIG. 7 illustrates current density as a function of time for one testing of the present disclosure occurring on Apr. 13, 2017. In this test, a radio translucent (RT) electrode was used. A hydrogenase was immobilized on the RT electrode. In some tests the hydrogenase was immobilized using a standard membrane and in other tests it was immobilized using a high sensitivity membrane.

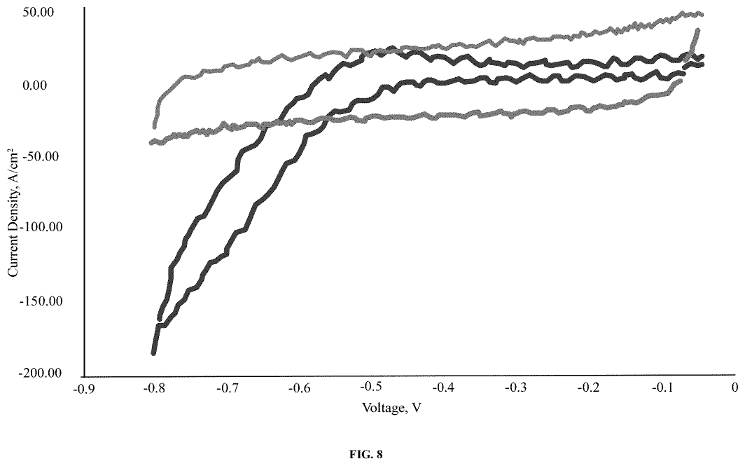

FIG. 8 illustrates current density as a function of voltage for one testing of the present disclosure. In this test, the pH of the system was maintained in the acidic range, to determine the impact of pH on the immobilized hydrogenase.

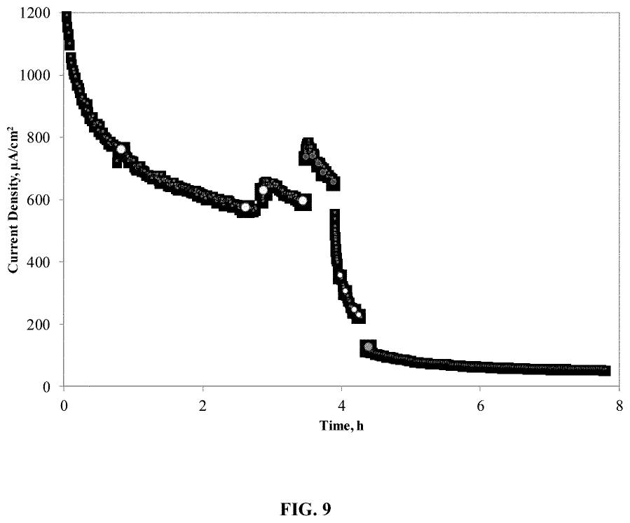

FIG. 9 illustrates current density as a function of time for one testing of the present disclosure occurring on Apr. 28, 2017. In this test, a carbon felt (CF) electrode was used. A hydrogenase was immobilized on the CF electrode using an electroconductive matrix. Here, the pH of the system was decreased by adding a mineral acid or strong acid, such as sulfuric acid (H₂SO₄) to the system. As indicated by FIG. 9, reducing the pH over several hours resulted in the current density decreasing as well.

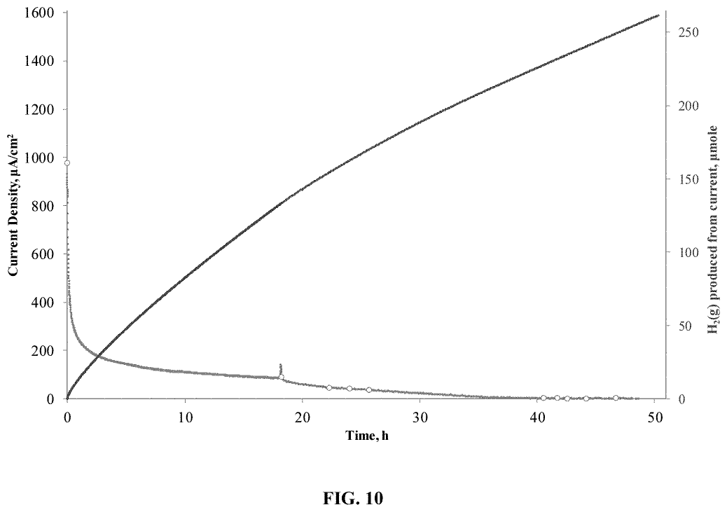

FIG. 10 illustrates current density as a function of time for one testing of the present disclosure. In this test, a CF electrode was used and the hydrogenase was immobilized on the electrode. As shown in FIG. 10, as the current density decreased the hydrogen produced by the system increases. The slight jump in current density at about 18 minutes appeared to be the result of bubbles appearing the CF electrode. They were removed with strong bubbling from argon (Ar) gas. Argon is an inert gas and other inert gases could be used.

The above examples demonstrate the production of hydrogen gas by a purified algal enzyme immobilized onto carbon felt. The enzyme remained highly active for at least 4 hours by addition of an acidic solution at various points during the process. Significant oxygen gas concentrations were not detected in the electrode chambers, which eliminated oxygen inactivation as a factor.

While various aspects and embodiments have been discussed above, those of skill in the art will recognize certain modifications, permutations, additions, and sub combinations thereof. It is therefore intended that the following appended claims and claims hereafter introduced are interpreted to include all such modifications, permutations, additions, and sub-combinations as are within their true spirit and scope.

### EXAMPLES

### Method Examples

1. A method comprising depositing a hydrogenase on an electrode, immobilizing the hydrogenase on the electrode using a matrix, supplying an electric current through the electrode to the hydrogenase, adding acid to the hydrogenase, and supplying an inert gas to the hydrogenase.
2. The method of Example 1, wherein the electrode is a carbon electrode.
3. The method of Example 2, wherein the carbon electrode consists substantially of glassy carbon, pyrolytic graphite, or carbon felt.
4. The method of Example 2, wherein the carbon electrode is radio translucent.
5. The method of Example 1, wherein the hydrogenase is a purified algal hydrogenase.
6. The method of Example 5, wherein the purified algal hydrogenase is from Chlamydomonas reinhardtii.
7. The method of Example 1, wherein the matrix is electroconductive.
8. The method of Example 1, wherein the acid is a mineral acid.
9. The method of Example 8, wherein the mineral acid is sulfuric acid.
10. The method of Example 1, wherein the hydrogenase is maintained at a constant pH level from the adding acid to the hydrogenase.
11. The method of Example 1, wherein the inert gas is argon.
12. The method of Example 1, wherein a biofilm is also used to immobilize the hydrogenase.
13. The method of Example 1, wherein the immobilizing of the hydrogenase enzyme using a matrix occurs prior to the depositing of the hydrogenase on the electrode.
14. The method of Example 1, wherein the matrix maintains the hydrogenase in close association with the electrode.
15. The method of Example 1, wherein the matrix is in direct contact with the electrode.
16. The method of Example 15, wherein the hydrogenase is not in direct contact with the electrode.
17. The method of Example 1, wherein the matrix is comprised of alginate or other conductive polymers.
18. The method of Example 1, further comprising anaerobically stabilizing the hydrogenase for a period of time prior to the supplying an electric current through the electrode to the hydrogenase.
19. The method of Example 18, wherein the period of time is less than one hour.
20. The method of Example 1, wherein the electric current supplied to the hydrogenase through the carbon electrode has a voltage of 6.5 to 7.5 V.
21. The method of Example 1, wherein the acid is a strong acid.
22. The method of Example 21, wherein the strong acid is hydrochloric acid.

### Device Examples

1. A device comprising an electrode, a hydrogenase immobilized directly on the electrode, a matrix encapsulating the hydrogenase, and a chamber, wherein the hydrogenase, the matrix, and the electrode are positioned within the chamber, the electrode is attached to a power source, and the chamber has at least one vent.
2. The device of Example 2, wherein the electrode is a carbon electrode.
3. The device of Example 3, wherein the carbon electrode consists substantially of glassy carbon, pyrolytic graphite, or carbon felt.
4. The device of Example 3, wherein the carbon electrode is radio translucent.
5. The device of Example 1, wherein the hydrogenase is purified algal hydrogenase.
6. The device of Example 5, wherein the purified algal hydrogenase is from Chlamydomonas reinhardtii.
7. The device of Example 1, wherein the matrix is electroconductive.
8. The device of Example 7, wherein the matrix is comprised of alginate or other conductive polymers.
9. The device of Example 1, wherein the acid is a mineral acid.
10. The device of Example 9, wherein the mineral acid is a strong acid.
11. The device of Example 9, wherein the mineral acid is sulfuric acid.
12. The device of Example 1, wherein the inert gas is argon.
13. The device of Example 1, wherein the acid is a strong acid.
14. The device of Example 13, wherein the strong acid is hydrochloric acid.

What is claimed is:

1. A method comprising:
depositing a hydrogenase on an electrode;
immobilizing the hydrogenase on the electrode using a matrix;
supplying an electric current through the electrode to the hydrogenase;
adding acid to the hydrogenase; and
supplying an inert gas to the hydrogenase.

2. The method of claim 1, wherein the electrode is a carbon electrode.

3. The method of claim 2, wherein the carbon electrode consists substantially of glassy carbon, pyrolytic graphite, or carbon felt.

4. The method of claim 2, wherein the carbon electrode is radio translucent.

5. The method of claim 1, wherein the matrix is electroconductive.

6. The method of claim 1, wherein the immobilizing of the hydrogenase enzyme using a matrix occurs prior to the depositing of the hydrogenase on the electrode.

7. The method of claim 1, further comprising anaerobically stabilizing the hydrogenase for a period of time prior to the supplying an electric current through the electrode to the hydrogenase.

8. The method of claim 1, wherein the period of time is less than one hour.

9. A device comprising:
an electrode;
a hydrogenase immobilized directly on the electrode;
a matrix encapsulating the hydrogenase; and
a chamber; wherein:
the hydrogenase, the matrix, and the electrode are positioned within the chamber,
the electrode is attached to a power source, and
the chamber has at least one vent.

10. The device of claim 9, wherein the matrix is electroconductive.

11. The method of claim 9, wherein the matrix is in direct contact with the electrode.

12. The method of claim 9, wherein the hydrogenase is not in direct contact with the electrode.

13. The device of claim 9, wherein the electrode is a carbon electrode.

14. The device of claim 10, wherein the carbon electrode consists substantially of glassy carbon, pyrolytic graphite, or carbon felt.

15. The device of claim 10, wherein the carbon electrode is radio translucent.
